(19) United States
(12) Patent Application Publication
(10) Pub. No.: US 2024/0358308 A1
(43) Pub. Date: Oct. 31, 2024

(54) SYSTEM FOR CAPACITIVELY CAPTURING ELECTRICAL BIOSIGNALS FROM A BIOSIGNAL SOURCE AND ASSOCIATED METHOD

(71) Applicants: Probiometrics LTD., Turnbridge Wells, Kent (GB); Capical GmbH, Braunschweig (DE)

(72) Inventors: Ralph Lloyd-Roberts, Tunbridge Wells, Kent (GB); Martin Oehler, Braunschweig (DE)

(21) Appl. No.: 18/688,470

(22) PCT Filed: Aug. 31, 2022
(86) PCT No.: PCT/GB2022/052219

§ 371 Date: Mar. 1, 2024

(30) Foreign Application Priority Data
Sep. 1, 2021 (GB) 2112463.1

(51) Int. Cl. A61B 5/277; A61B 5/00; A61B 5/01; A61B 5/0205; A61B 5/27; A61B 5/282; A61B 5/308

(52) U.S. Cl. CPC A61B 5/277; A61B 5/01; A61B 5/02055; A61B 5/27; A61B 5/282; A61B 5/308; A61B 5/6887; A61B 5/7203; A61B 5/7221; A61B 5/7225; A61B 2562/0214; A61B 2562/0271; A61B 2562/046; A61B 2562/164; A61B 2562/227

(57) ABSTRACT

A system is disclosed for capacitively capturing electrical biosignals from a biosignal source in a medical care screening or monitoring environment. The system comprises: a sheet; two capacitive electrodes supported by the sheet: a printed circuit board, PCB, supported by the sheet; two stretchable contacts respectively arranged between each capacitive electrode and the PCB, wherein each stretchable contact is configured to stretch to permit relative movement between its respective capacitive electrode and the PCB while maintaining electrical contact between the PCB and its respective capacitive electrode.

### TECHNICAL FIELD

The present disclosure relates to system for capacitively capturing electrical biosignals from a biosignal source, such as a human, and associated method.

### BACKGROUND

Existing systems for measuring the heart rate of a person lying in a bed generally include placing a sensor system under a mattress of the bed and detecting the ballistocardiogram and body movements using piezo sensors or pressure sensors. Other examples include using a radar sensor or camera beside or above a bed to detect movements of a person lying in the bed. These systems have limited precision when it comes to heart rate detection and beat-to-beat interval detection and do not allow an ECG reading to be performed.

An alternative means by which to provide an ECG reading with improved accuracy is for a person to wear an ECG monitoring device on their chest, fixed in place either with a strap or an adhesive patch, while sleeping. However, this also gives rise to the problem of reduced sleep comfort.

Existing systems also generally have limited range of applicability. For example, existing systems for measuring heart rate of a person lying in a bed that do not involve a person physically wearing a device generally can't be used in any other context or can only be used in very limited contexts.

As can be seen, existing systems for measuring heart rate of a person lying in a bed suffer from significant drawbacks in terms of accuracy and/or user comfort. Additionally, they can often only be used in very limited contexts. It would be advantageous to provide systems and methods which address one or more of these problems, in isolation or in combination.

### Overview

This overview introduces concepts that are described in more detail in the detailed description. It should not be used to identify essential features of the claimed subject matter, nor to limit the scope of the claimed subject matter.

According to one aspect of the present disclosure, there is provided a system comprising a sheet, two capacitive electrodes supported by the sheet, a printed circuit board, PCB, supported by the sheet, two stretchable contacts respectively arranged between each capacitive electrode and the PCB. Each stretchable contact is configured to stretch to permit relative movement between its respective capacitive electrode and the PCB while maintaining electrical contact between the PCB and its respective capacitive electrode.

In one example, the system may be suitable for capacitively capturing electrical biosignals from a biosignal source, for example, in a medical care screening or monitoring environment. Other or alternative environments could include one or more of: health monitoring, sleep monitoring, fitness monitoring, employee monitoring, gamer monitoring and/or consumer research subject monitoring.

The provision of stretchable contacts between the PCB and the capacitive electrodes provides for a comfortable and robust sensing system. Relative movement of components supported by the sheet, for example the PCB and the capacitive electrodes, is permitted by the stretchable contacts. This reduces restriction of movement of the sheet caused by the components the sheet supports, thereby increasing comfort as well as the ability of the sheet to conform to different surfaces. In this way, the sheet is both more comfortable for a user while also being able to be used in a variety of contexts.

Further, as the sensing components are provided on a sheet, the sensing system can be placed onto any surface and used for capturing electrical biosignals from a biosignal source, such as a person, lying on or leaning against the surface on which the sheet is placed. In this manner, any surface, such as a bed or examination table, can be provisioned with biosignal sensing capabilities.

The sheet can comprise a textile material. This means that the sheet has an inherent degree of flexibility and can conform to the contours of the surface on to which it is placed.

The sheet can be a bed sheet. In this manner, the sheet can be placed on the mattress of a bed, in place of an ordinary bedsheet or between a mattress and ordinary bedsheet, to provision the bed with biosignal sensing capabilities.

At least one of the stretchable contacts can be substantially planar or flat in profile. In this way, the contribution of the of the contacts to the overall profile of the system is reduced, further improving the level of comfort afforded by the system when a user lies on or leans against the sheet.

At least one of the stretchable contacts can comprise a thermoplastic polyurethane substrate. The use of a thermoplastic polyurethane substrate can assist in enabling the stretchable contacts to stretch.

The at least one of the stretchable contacts can comprise a conductive trace supported by the thermoplastic polyurethane substrate. The conductive trace could equally be any suitable conduit for electrical current, such as a wire or ink, e.g. silver or copper ink.

The conductive trace can comprise copper and/or silver. These metals conduct electricity while also being ductile, which is beneficial for use in a stretchable contact.

The conductive trace can comprise a serpentine portion. This can assist in enabling the stretchable contact to stretch while maintaining an electrical connection between terminals of the electrical contact as stretching of the substrate causes the serpentine portion to straighten out without breaking.

At least one of the stretchable contacts can comprise a first contact region coupled to its respective capacitive electrode. For example, the contact region can have a planar profile. In this way, the contribution of the of the contacts to the overall profile of the system is reduced, further improving the level of comfort afforded by the system when a user lies on or leans against the sheet.

The coupling between the first contact region and the respective capacitive electrode can comprise a bond created by melting a/the TPU substrate of the at least one of the stretchable contacts. This creates a low profile, reliable, bond without the need for any crimped components, which would reduce user comfort when lying on or leaning against the sheet.

The first contact region can comprise a conductive planar surface.

The first contact region can be circular in shape. This can assist in manufacture or assembly of the system as it is easier to line up the centre of the contact region with the desired contact location as the centre of the contact region is more easily identifiable.

At least one of the stretchable contacts can comprise a second contact region coupled to the PCB.

The coupling between the second contact region and the PCB can comprise a bond created by melting a/the TPU substrate of the at least one of the stretchable contacts.

The second contact region can comprise a conductive planar surface.

The second contact region can be circular in shape.

At least one of the capacitive electrodes can comprise first and second layers of an electrically conductive textile material separated by a layer of electrically insulating textile material.

The use of such electrodes improves the level of comfort afforded by the system when a user lies on or leans against the sheet. The use of textile materials improves comfort when a user lies on the electrode as the conform both to the surface upon which the sheet is placed as well as to the user, for example where the sheet is placed on a mattress and the user lies on the sheet causing both the mattress and the sheet to conform to the user. The ability of the textile materials used in the capacitive electrodes to conform to the user also enables a better biosignal reading to be obtained as contact area with the user is increased when the user is lying on the sheet. Additionally, when the sheet is placed on an object, such as a bed, the textile materials of the at least one of the capacitive electrode can conform to the object.

At least one of the capacitive electrodes can be flat or have a planar profile. Again, this improves user comfort, for example, when the sheet is placed on a bed or other surface and a user lies on the sheet.

A cutaway can be provided on the at least one of the capacitive electrodes. The cutaway can comprise a region where the first layer of the electrically conductive textile material and the electrically insulating textile material have been removed. The respective stretchable contact can be electrically connected to the second layer of the electrically conductive textile material via the cutaway. The stretchable contact can also be electrically connected to the first layer of the electrically conductive textile material, for example, on the same side of the capacitive electrode.

In this manner, the stretchable contact can electrically connected to both conductive textile materials of the at least one of the capacitive electrodes on the same side of the capacitive electrode, thereby reducing overall profile of the stretchable contact and the capacitive electrode. This further improves comfort for the user lying on or leaning against the sheet.

Each of the two capacitive electrodes can be arranged either side of a longitudinal axis of the sheet. Additionally or alternatively, each of the two capacitive electrodes can be arranged toward one end of the sheet along the longitudinal axis of the sheet. These arrangements improve the quality of electrical biosignals obtained from a biosignal source lying on or leaning against the sheet as it ensures the electrodes are in the locations required to obtain, for example, an ECG signal.

The system can comprise three, four or more capacitive electrodes. Additionally or alternatively, the three or four capacitive electrodes can be arranged in pairs either side of a longitudinal axis of the sheet. Additionally or alternatively, each of the four capacitive electrodes can be arranged toward one end of the sheet along the longitudinal axis of the sheet.

Using three or four capacitive electrodes further improves the quality of the obtained electrical biosignals from a biosignal source lying on or leaning against the sheet. For example, different heart axes can be covered by the electrodes, where more than two capacitive electrodes are used (e.g. three or more) and in contact with the biosignal source to select a pair of electrodes which has the largest biosignal amplitude

The PCB can comprise a flexible polymer substrate. The use of such a PCB further improves comfort for a user lying on or leaning against the sheet as the PCB is able to conform to the surface upon which the sheet is placed and also to the user.

The PCB can comprise a thermoplastic polyurethane substrate. In this arrangement, the use of a thermoplastic polyurethane substrate can assist in enabling the PCB to stretch, much like the stretchable contacts, giving rise to improved comfort as restrictions on movement on the sheet by components supported by the sheet is further reduced.

The system can further comprise an electronic signal processing system connected to each capacitive electrode via the PCB and each electrode's respective stretchable contact. The electronic signal processing system can be configured to process biosignals detected by the capacitive electrodes, for example, to output an ECG signal/reading for the user.

The detected biosignals can comprise ECG signals.

The system can further comprise a temperature sensor supported by the sheet and in electrical contact with the PCB. In this arrangement, temperature readings can be provided in conjunction with the biosingals obtained via the capacitive electrodes.

The temperature sensor can comprise an array of temperature sensors. This means that temperature readings can be taken at discrete locations to provide a more detailed output of biosignal source/user temperature.

The two capacitive electrodes can be of the same size. Having electrodes of the same or similar size allows for efficient common noise reduction when processing detected biosignals.

The PCB and two stretchable contacts can comprise a single substrate. In other words, they can be formed from a single substrate and be provided as a single integrated unit. In this way, the entire PCB and stretchable contact arrangement can be stretchable. This further improves comfort for a user lying on the sheet.

The single substrate can comprise a thermoplastic polyurethane substrate. This can permit stretching of the single substrate. The PCB and stretchable contact arrangement could further comprise serpentine conductive traces to further aid in facilitating stretching of the single substrate.

A sheet is considered to be any sheet of a material. A sheet could be considered to be any sheet of material suitable for covering or laying on another object, such as a bed, examination table, examination table, chair or other object. A sheet could be considered to be a material substrate which is relatively thin when compared to its planar extent. A sheet could be considered to be a piece of material with a planar profile and/or a sheet could be considered to be a flat piece of material.

Flexible generally refers to something which is capable of being bent without breaking.

Stretchable generally refers to something which can be stretched without breaking.

A biosignal source can be any living being, in particular a human. Examples of biosignals include ECG signals, EEG signals and similar signals that are able to be captured electrically. Biosignals can be evaluated, e.g. for medical diagnostic purposes, or for any other desired purposes such as health monitoring, sleep monitoring, fitness monitoring, employee monitoring, gamer monitoring and/or consumer research subject monitoring.

### BRIEF DESCRIPTION OF THE FIGURES

Illustrative implementations of the present disclosure will now be described, by way of example only, with reference to the drawings. In the drawings:

FIG. 1 shows a schematic representation of a system for capacitively capturing electrical biosignals from a biosignal source according to the present disclosure;

FIG. 2 shows a further schematic representation of the system for capacitively capturing electrical biosignals from a biosignal source according to the present disclosure;

FIG. 3 shows a schematic representation of a PCB, a stretchable contact and a capacitive electrode according to the present disclosure;

FIG. 4 shows a representation of the PCB, two stretchable contacts and two capacitive electrodes according to the present disclosure; and

FIG. 5 shows a method of capacitively capturing electrical biosignals from a biosignal source according to the present disclosure.

Throughout the description and the drawings, like reference numerals refer to like features.

### DETAILED DESCRIPTION

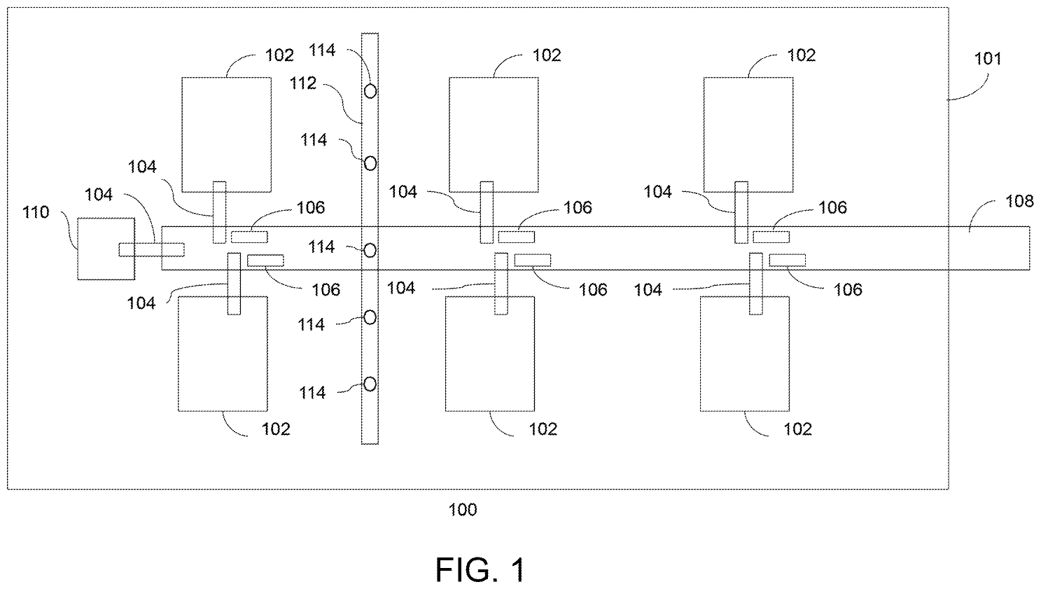

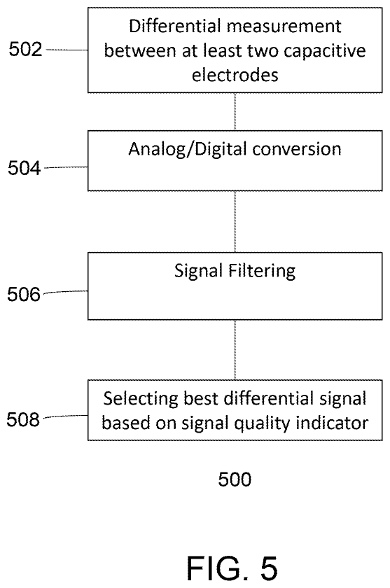

This detailed description describes, with reference to FIGS. 1, 2, 3 and 4, a system for capacitively capturing electrical biosignals from a biosignal source comprising a PCB, a stretchable contact and a capacitive electrode. Finally, a method of capacitively capturing electrical biosignals from a biosignal source is described with reference to FIG. 5.

The system disclosed herein relates generally to a system for capacitively capturing electrical biosignals from a biosignal source. By way of example, a biosignal source can be any living being, in particular a human. By way of example, ECG signals, EEG signals and similar signals that are able to be captured electrically, for example using capacitive electrodes, can be recorded as biosignals. The biosignals can be evaluated, e.g. for medical diagnostic and other purposes.

A schematic representation of a system 100 for capacitively capturing electrical biosignals from a biosignal source is shown in FIG. 1. The system 100 comprises a sheet 101 which supports capacitive electrodes 102. Each capacitive electrode 102 is electrically connected to a central printed circuit board (PCB) 108 via a respective stretchable contact 104. Electrical components 106 are also provided on the PCB 108. A further driven electrode 110 is also supported by the sheet 101 and electrically connected to the PCB 108 via a respective stretchable contact 104. A temperature sensor 112 is also supported by the sheet 101 and electrically connected to the PCB 108.

The sheet 101 can, in some examples, be a bed sheet for placing on a mattress of a bed. The sheet could also be a cover for a cushion, pillow, chair, mattress or any other object. For example, the sheet 101 could be used as a cover for one or more of an operating table (e.g. for use in an operating theatre), an examination table or chair (e.g. for use in a GP surgery), a care home bed, couch, or a lumbar cushion. The sheet could also be suitable for laying over of covering a range of objects. Advantageously, this means that these objects can retrospectively be provisioned with biosignal sensing capabilities via use of the sheet 101.

The sheet 101 can, for example, be made from a textile material. The sheet 101 supports various components of the system 100. Components supported by the sheet, such as the capacitive electrodes 102, stretchable contacts 104, PCB 108, driven electrode 110 and temperature sensor 112 can, for example, be supported the sheet 101 by being adhered, or otherwise coupled, to a surface of the sheet 101. This can be achieved by using, for example, an adhesive. Alternatively, the supported components can be supported by the sheet 101 by forming an integral part of the sheet 101, for example, by being integrated into the sheet 101. For example, the sheet 101 can be manufactured such that some or all of the components supported by the sheet themselves form part of the sheet 101. For example, the capacitive electrodes 102 and/or the driven electrode 110 could provide a dual purpose of contributing to the surface area of the sheet while also assisting in capacitively capturing electrical biosignals from a biosignal source. For example, during manufacture of the system 100, sections of the sheet 101 could be removed and replaced with capacitive electrodes 102 and/or the driven electrode 110.

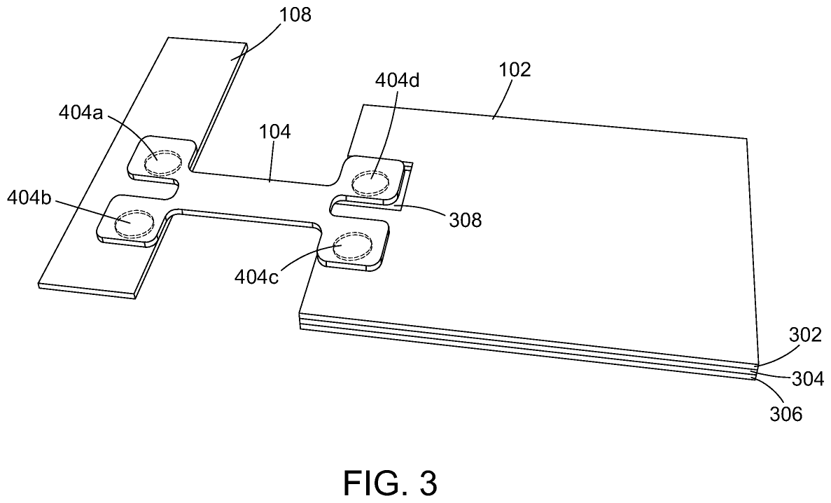

The capacitive electrodes 102 are configured to detect biosignals, such as ECG signals, from a biosignal source, such as a human lying on, or otherwise resting against, the sheet 101. The capacitive electrodes 102 comprise at least two layers of an electrically conductive material separated by at least one layer of insulating material. This can be seen more clearly in FIG. 3 which depicts a first electrically conductive layer 302 and a second electrically conductive layer 306 separated by an insulating layer 304. One or more of these layers can be made from a textile material. The capacitive electrodes 102 may be textile capacitive electrodes, meaning the layers forming the capacitive electrodes 102 are made from a textile material. In some examples, the sheet 101 could form the insulating layer 304 with the first electrically conductive layer 302 and the second electrically conductive layer 306 of each capacitive electrode 102 being adhered, or otherwise coupled, to either side of the sheet. The driven electrode 110 normally uses only one layer of conductive material, but also can have exactly the same structural features as the capacitive electrode 102 outlined above.

As depicted in FIG. 1, the capacitive electrodes 102 are arranged either side of the PCB 108 and along the length of the PCB 108. The capacitive electrodes 102 are located laterally either side of the PCB 108. This configuration ensures that, when a biosignal source, such as a human, is lying on or otherwise resting against the sheet 101, at least two capacitive electrodes 102 are in contact with the biosignal source so that a biosignal reading, such as an ECG reading, can be taken. Additionally, different heart axes are covered by the electrodes, where more than two capacitive electrodes 102 are used and in contact with the biosignal source to select a pair of electrodes which has the largest biosignal amplitude. In some examples, all of the capacitive electrodes 102 are all of the same size or a similar size to reduce external interferences due to the differential measurement. An ECG signal is always a differential measurement between two electrodes. Providing electrodes of the same or similar size allows for efficient common noise reduction.

In the example depicted in FIG. 1, six capacitive electrodes 102 are provided in two rows along the longitudinal length of the sheet 101. Alternatively, two capacitive electrodes 102 could be provided. These could be provided either side of the PCB 108 or along the longitudinal length of the sheet 101 on the same side of the PCB 108. Equally, three, four, five, seven, eight or more capacitive electrodes 102 could be provided. For example, four capacitive electrodes 102 may be arranged in pairs either side of a longitudinal axis of the sheet 101. Advantageously, this configuration enables the capacitive electrodes 102 to obtain a signal from a biosignal source, such as a human, when lying on or otherwise resting against the sheet 101 in a number of possible lying positions. In some examples, the capacitive electrodes 102 could be provided in three rows along the longitudinal length of the sheet 101.

In some examples, where the sheet 101 is a bed sheet, the arrangement of the capacitive electrodes 102 is such that they are located in a region of the bed sheet 101 where the chest of a person lying on the bed is likely to be. For example, the centre point of the capacitive electrodes 102 can be closer to one end of the sheet 101, in a longitudinal direction of the sheet 101, than the other.

The stretchable contacts 104 are configured to electrically connect the capacitive electrodes 102 to the PCB 108 such that biosignals detected by the capacitive electrodes 102 can be transmitted to the PCB 108. Each stretchable contact 104 is configured to stretch to permit relative movement between the PCB 108 and each stretchable contact's 104 respective capacitive electrode 102 while maintaining electrical contact between the PCB 108 and capacitive electrode 102. Advantageously, this permits relative movement between each capacitive electrode 102 and the PCB 108 giving rise to significant benefits in terms of comfort for a human lying on, or otherwise resting against, the sheet 101. Each stretchable contact 104 may be configured to stretch and permit relative movement between the PCB 108 and each stretchable contact's 104 respective capacitive electrode 102 in the plane of the sheet 101.

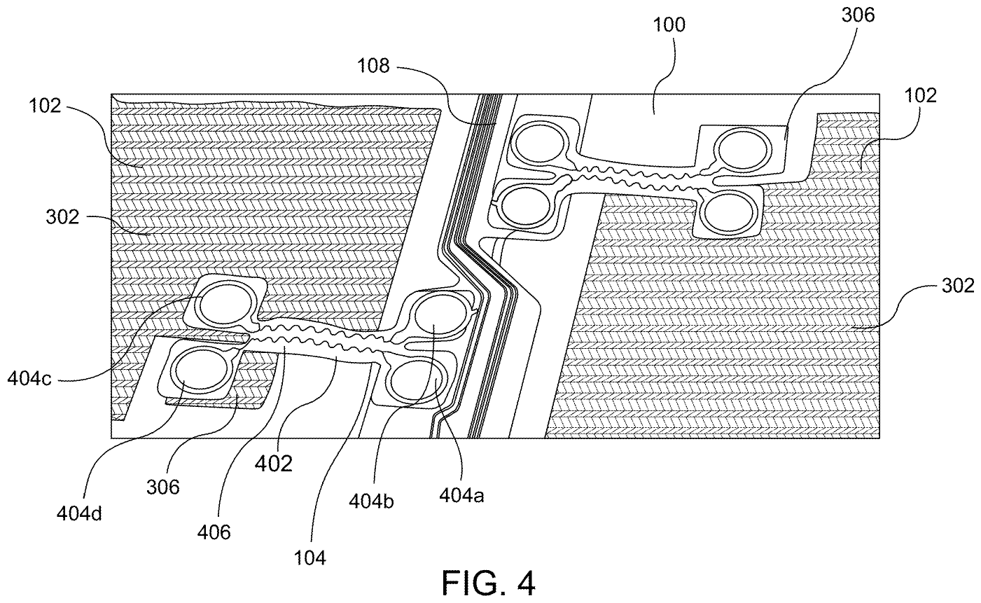

Example configurations of the stretchable contacts 104 are depicted in FIGS. 3 and 4. As can be seen in FIG. 4, each stretchable contact 104 is formed of a stretchable substrate 402. The substrate 402 could be, for example, a thermoplastic polyurethane (TPU) substrate which is capable of being stretched when the PCB 108 and capacitive electrode 102 move relative to one another. Alternatively, silicone substrates such as could be used.

Each stretchable contact 104 comprises four contact regions 404a, 404b, 404c, 404d supported by the stretchable substrate 402. At least a portion of the contact regions 404a, 404b, 404c, 404d may be exposed on one side to permit electrical contact with another component, as detailed below. One or more of the contact regions 404a, 404b, 404c, 404d may be made of a ductile conductive material, such as copper and/or silver (e.g. silver ink), to facilitate stretching of the stretchable contact 104 while maintaining an electrical connection between the PCB 108 and capacitive electrode 102. As can be seen in FIGS. 3 and 4, two contact regions 404a and 404b are coupled to the PCB 108 on a first side of the stretchable contact 104. At an opposing second side of the stretchable contact 104, a further contact region 404c is coupled to the first electrically conductive layer 302 of the stretchable contact's 104 respective capacitive electrode 102. A further contact region 404d is coupled to the second electrically conductive layer 306 of the stretchable contact's 104 respective capacitive electrode 102 via a cutaway 308. At the cutaway 308, a surface of the second electrically conductive layer 306 is exposed by removing a section of the first electrically conductive layer 302 and the insulating layer 304. This enables the contact region 404d to be coupled to the second electrically conductive layer 306 in a manner which reduces the overall profile of the stretchable contact 104 and respective capacitive electrode 102. This further improves comfort for a human lying on, or otherwise resting against, the sheet 101.

One or more of the contact regions 404a, 404b, 404c, 404d can be circular in shape. This facilitates easier placement of the contact regions 404a, 404b, 404c, 404d when adhering them to the PCB 108 or respective capacitive electrode 102 as the centre of the circular contact regions 404a, 404b, 404c, 404d can be used for easier alignment with the desired location.

As can be seen in FIG. 4, one or more of the contact regions 404a, 404b, 404c, 404d can comprise a circular outer perimeter. The one or more of the contact regions 404a, 404b, 404c, 404d can also comprise inwardly projecting portions projecting inwardly from the circular outer perimeter. In this manner, the amount of conductive material, such as copper, used can be reduced while ensuring adequate surface area for electrical contact is provided. Additionally, the structure helps ensure good adhesion between the contact regions 404a, 404b, 404c, 404d and the PCB 108 and/or the capacitive electrode 102.

One or more of the contact regions 404a, 404b, 404c, 404d can be coupled to the PCB 108 and/or the capacitive electrode 102 via a bond. The bond can be created by melting the substrate 402, for example at the location of the contact regions 404a, 404b, 404c, 404d, while the contact regions 404a, 404b, 404c, 404d are held or pressed against the component they are to be attached to. In the example where the substrate 402 comprises a thermoplastic polyurethane (TPU) substrate, the TPU could be melted at the location of the contact regions 404a, 404b, 404c, 404d to form a bond between the contact regions 404a, 404b, 404c, 404d and the PCB 108 and/or the capacitive electrode 102. In the example where the PCB also comprises a thermoplastic polyurethane (TPU) substrate, the TPU of both the stretchable contact 104 and the PCB can be melted at the location of the contact regions 404a, 404b, 404c, 404d to form a bond between the contact regions 404a, 404b, 404c, 404d and the PCB 108.

Alternatively, the PCB 108 and any or all stretchable contact(s) 104 can be made out of a single substrate, for example, they can be provided as a single stretchable substrate such as a thermoplastic polyurethane (TPU) substrate.

Each stretchable contact 104 further comprises a conductive connection 406, as can be seen in FIG. 4, supported by the stretchable substrate 402. This could, for example, take the form of a conductive trace. It could also be a wire or any conductive connection. The conductive connection 406 may be made of a ductile conductive material, such as copper and/or silver (e.g. silver ink), to facilitate stretching of the stretchable contact 104 while maintaining an electrical connection between the PCB 108 and capacitive electrode 102. Additionally, or alternatively, the conductive connection 406 may comprise one or more serpentine portions, again to facilitate stretching of the stretchable contact 104 while maintaining an electrical connection between the PCB 108 and capacitive electrode 102.

As can be seen in FIG. 4, the conductive connection 406 may comprise a first serpentine trace connecting contact regions 404a and 404c and a second serpentine trace connecting contact regions 404b and 404d, all supported by the stretchable substrate 402. Alternatively, the conductive connection 406 may comprise a first pair of serpentine traces connecting contact regions 404a and 404c and a second pair of serpentine traces connecting contact regions 404b and 404d, all supported by the stretchable substrate 402. Further traces can be provided between contact regions 404a and 404c and contact regions 404b and 404d. It is understood that at least one serpentine traces could be provided between contact regions 404a and 404c and/or contact regions 404b and 404d.

Electrical components 106 are also provided on the PCB 108. The main function of these is to provide impedance conversion and optional amplification of the biosignal. For capacitive electrodes 102, a very high input impedance is necessary (usually larger than 1 GOhm). For example, field-effect transistor (FET) based amplifiers are suitable for this impedance conversion as they provide a very high input impedance. The aim is to provide an impedance conversion with a very high input impedance for the or each capacitive electrode 102 and a low output impedance to reduce noise on cables connecting the capacitive electrode 102 to the control unit 201. Optional additional electrical components 106 include resistors and/or diodes that can be used for a defined bias path at the input. Optionally, electrostatic discharge (ESD) protection can also be implemented using an ESD protection device such as one or more of Zener diodes, varistors, SCR (Silicon Controlled Rectifier), and TVS (Transient Voltage Suppressor) components.

As can be seen in FIG. 1, the PCB 108 extends along the sheet 101 in a longitudinal direction of the sheet 101. In some examples, the PCB lies along a central longitudinal axis of the sheet 101. In some examples, the PCB 108 can be a flex PCB. For example, the PCB can be made from a flexible polymer substrate, such as a polyimide film. Additionally, or alternatively, the PCB 108 can have a serpentine shape or serpentine or portions along its length to further facilitate flexibility of the PCB 108. In some examples, the PCB 108 can comprise a stretchable substrate, such as a thermoplastic polyurethane substrate. In this manner, the PCB can be stretchable as well as flexible.

As mentioned above, the driven electrode 110 normally consists of only one conductive layer, providing a capacitive reference potential, which capacitively drives the human body, or other biosignal source body, to the system reference potential. The electric potential is controlled by the control unit 201.

Alternatively, the driven electrode 110 can have exactly the same structural features as the capacitive electrodes 102 outlined above. In other words, the driven electrode 110 can comprise a first electrically conductive layer and a second electrically conductive layer separated by an insulating layer with any of the features specified in relation to the capacitive electrodes 102.

The driven electrode 110 is electrically connected to the PCB via a stretchable contact 104. The driven electrode 110 is configured to produce a signal to control the body potential (electrical potential at the surface of the human or other biosignal source body) as close as possible to a reference potential (e.g. ground potential) of the control unit 201.

The temperature sensor 112 comprises one or more discrete sensors 114 located along the length of the temperature sensor 112. These sensors 114 are configured to provide temperature readings of the biosignal source, such as a human lying on, or otherwise resting against, the sheet 101, at various locations. Temperature sensors with a small component package (e.g. quad-flat no-leads, ball grid array etc.) and/or a thin package (less than 1 mm) can be used in some example implementations.

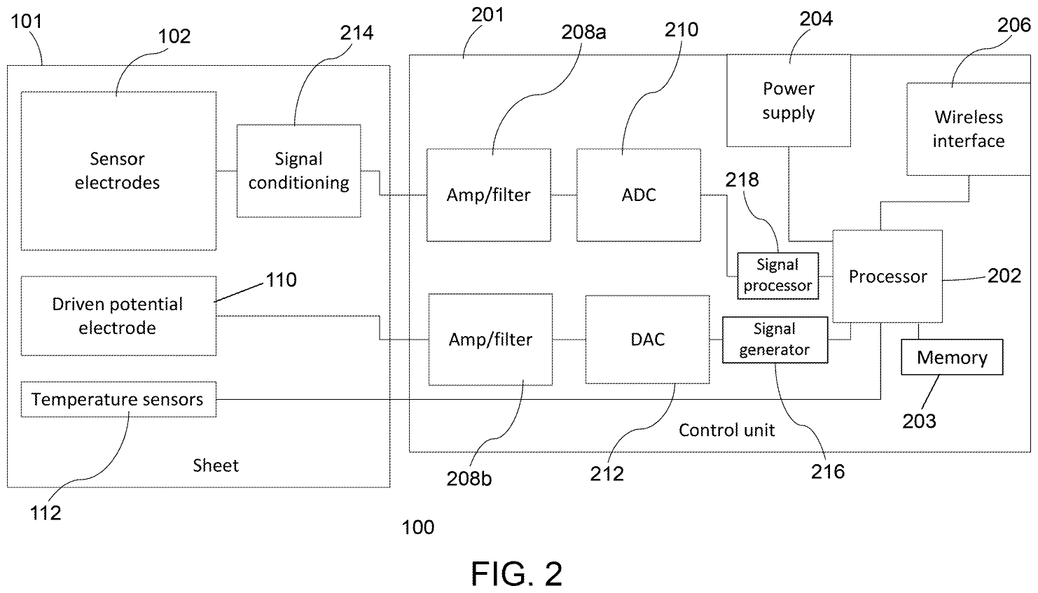

FIG. 2 shows a further schematic and further simplified representation of the system 100 for capacitively capturing electrical biosignals from a biosignal source. This time a control unit 201 is shown in addition to the sheet 101. The sheet 101 further includes a signal conditioning module 214 which is the same component as the aforementioned electrical components 106 and provides the same functionality described above. The control unit 201 is depicted in FIG. 2 as being separate from sheet 101 and the components of the sheet and is in communication with the electrical components of the sheet 101 via a wired connection. Alternatively, an interface 206 could be used in place of the depicted wired connection. In an alternative arrangement, control unit 201 may be provided on, or otherwise supported by, sheet 101.

The control unit 201 functions as an electronic signal processing system connected to each capacitive electrode 102 via the PCB 108 and each electrode's respective stretchable contact 104. The control unit 201 is configured to process biosignals detected by the capacitive electrodes 102. The control unit 201 comprises a processor 202 (in particular a hardware processor), a memory 203, a power supply 204, a wireless interface 206, two amplifier/filter modules 208a and 208b. The first amplifier/filter module 208a is connected to the capacitive electrodes 102 and the second amplifier/filter module 208b is connected to the driven electrode 110. The control unit 201 further comprises an analog-to-digital converter (ADC) 210, connected between the first amplifier/filter module 208a and the processor 202, and a digital-to-analog converter (DAC) 212, connected between the second amplifier/filter module 208b and the processor 202. The control unit 201 also comprises a signal generator 216, for providing a signal to the driven electrode 110, and a signal processor 218, for processing signals received from the capacitive electrodes 102.

The processor 202 is depicted as being coupled directly or indirectly to every component of the system. Alternatively, the processor 202 could be coupled to a central bus structure with all other components also connected either directly or indirectly to the central bus structure.

In operation, the processor 202 of control unit 201 executes a computer program comprising computer-executable instructions that may be stored in memory 203. When executed, the computer-executable instructions may cause the control unit 201 and system 100 to perform one or more of the methods described herein. The results of the processing performed may be displayed to a user via a display adapter and display device (not depicted) which may comprise part of the control unit 201 or may be separate from the control unit 201 and in communication with the control unit 201. Such a display device may be connected to the control unit 201 via, for example, a network such as the internet. User inputs for controlling the operation of the control unit 201 and system 100 may be received via user-input device adapters from a user-input device, such as a keyboard, graphical user interface (GUI), and/or a mouse (also not depicted). User inputs for controlling the operation of the control unit 201 and system 100 may also be received from user-input device separate from the control unit 201 and in communication with the control unit 201, such as a keyboard, graphical user interface (GUI), and/or a mouse (also not depicted). Such a user-input device may be connected to the control unit 201 via, for example, a network such as the internet.

The power supply 204 can take the form of a means for connecting to mains power, such as a plug. Alternatively, the power supply 204 can comprise one or more batteries for powering the system 100.

Although a wireless interface 206 is depicted, interface 206 could alternatively be any interface suitable for communicating with one or more other computer systems or networks. For example, a wired interface could be provided.

The first amplifier/filter module 208a is connected to the capacitive electrodes 102 and is configured to pre-amplify the biosignal and remove unwanted frequency components (e.g. noise, interferences). It also limits the bandwidth to the input bandwidth of the analog-to-digital converter (ADC) 210.

The second amplifier/filter module 208b is connected to the driven electrode 110 and is configured to amplify and drive the signal for the driven electrode. It also limits the maximum current to the driven electrode.

The analog-to-digital converter (ADC) 210 is configured to convert analog signals detected by the capacitive electrodes 102 via the first amplifier/filter module 208a to digital signals for processing by the processor. Input bandwidth of the ADC is set to a suitable input bandwidth for ECG signals. For example, a minimum bandwidth of 250 Hz could be used or a bandwidth of 1 kHz.

The digital-to-analog converter (DAC) 212 is configured to convert digital signals provided by the processor 202 into analog signals transmitted to the driven electrode 110 via the second amplifier/filter module 208b. This signal is a reference signal provided by the processor to reduce common mode noise.

The signal generator 216 configured to provide a signal to the driven electrode 110. The generated signal is converted to an analog signal by the digital-to-analog converter (DAC) 212 and then passed on to the driven electrode 110 via the second amplifier/filter module 208b.

The signal processor 218 is configured to receive signals from the capacitive electrodes 102 via the signal conditioning module 214, first amplifier/filter module 208a and the analog-to-digital converter (ADC) 210. The signal processor 218 may be configured to combine capacitive electrodes 102 to differential pairs to generate ECG signals. The best differential pairs are selected for further processing.

It will be apparent from the above description of the system 100 depicted in FIGS. 1, 2, 3 and 4 that the configuration of the sheet 101 and components on the sheet results in a system 100 for capacitively capturing electrical biosignals from a biosignal source which gives rise to an improved level of comfort for anyone lying on the sheet 101 while also increasing the accuracy of biosignal, such as ECG signal, detection. This is a result of the properties of the various components described above. Relative movement between the various components is permitted which has advantages over sensors that are provided in a fixed relationship to one another. The fact that some components are flexible and/or stretchable contributes to user comfort.

The method disclosed herein relates generally to a method of capacitively capturing electrical biosignals from a biosignal source. This method is depicted in FIG. 5 and can be carried out, for example, using the system depicted in FIGS. 1, 2, 3 and 4.

At block 502, a differential measurement between at least two of the capacitive electrodes 102 is taken. This step comprises producing a reference potential at the driven electrode 110 to stabilise the system. A differential measurement is then taken between at least two of the capacitive electrodes 102.

At block 504, the detected signals are converted from analog to digital by analog-to-digital converter (ADC) 210. The ADC resolution is 8 bit or higher (for example, 16-bit or 24-bit) and the sampling rate is 250 Hz or higher (preferably 1 kHz).

At block 506, the signals are filtered. This may include bandpass-filtering to remove non biosginal related signal components and/or notch-filtering for power line noise reduction (e.g. 50/60 Hz).

At block 508, the best signal is selected based on a determined signal quality indicator for each signal. These signal quality indicators may include signal amplitude indicators, noise indicators, periodicy indicators or other indicators which are specific for the biosignal signal. The best signal selection is done continuously on the input data to ensure best output data even if the subject has changed their lying position during the usage of the sheet.

The above detailed description describes a variety of exemplary arrangements of and methods of using a control mechanism. However, the described arrangements and methods are merely exemplary, and it will be appreciated by a person skilled in the art that various modifications can be made without departing from the scope of the appended claims. Some of these modifications will now be briefly described, however this list of modifications is not to be considered as exhaustive, and other modifications will be apparent to a person skilled in the art.

While various specific combinations of components and method steps have been described, these are merely examples. Components and method steps may be combined in any suitable arrangement or combination. Components and method steps may also be omitted to leave any suitable combination of components or method steps. The ordering of method steps may also be altered and steps need not necessarily be carried out in the described order.

The described methods may be implemented using computer executable instructions. A computer program product or computer readable medium may comprise or store the computer executable instructions. The computer program product or computer readable medium may comprise a hard disk drive, a flash memory, a read-only memory (ROM), a CD, a DVD, a cache, a random-access memory (RAM) and/or any other storage media in which information is stored for any duration (e.g., for extended time periods, permanently, brief instances, for temporarily buffering, and/or for caching of the information). A computer program may comprise the computer executable instructions. The computer readable medium may be a tangible or non-transitory computer readable medium. The term “computer readable” encompasses “machine readable”.

The singular terms “a” and “an” should not be taken to mean “one and only one”. Rather, they should be taken to mean “at least one” or “one or more” unless stated otherwise. The word “comprising” and its derivatives including “comprises” and “comprise” include each of the stated features, but does not exclude the inclusion of one or more further features.

The above implementations have been described by way of example only, and the described implementations are to be considered in all respects only as illustrative and not restrictive. It will be appreciated that variations of the described implementations may be made without departing from the scope of the disclosure. It will also be apparent that there are many variations that have not been described, but that fall within the scope of the appended claims.

1. A system for capacitively capturing electrical biosignals from a biosignal source in a medical care screening or monitoring environment, the system comprising:
a sheet;
two capacitive electrodes supported by the sheet;
a printed circuit board, PCB, supported by the sheet;
two stretchable contacts respectively arranged between each capacitive electrode and the PCB,
wherein each stretchable contact is configured to stretch to permit relative movement between its respective capacitive electrode and the PCB while maintaining electrical contact between the PCB and its respective capacitive electrode.

2. The system of claim 1, wherein the sheet comprises a textile material.

3. The system of claim 1 or claim 2, wherein the sheet is a bed sheet.

4. The system of any preceding claim, wherein at least one of the stretchable contacts comprises a thermoplastic polyurethane substrate.

5. The system of claim 4, wherein the at least one of the stretchable contacts comprises a conductive trace supported by the thermoplastic polyurethane substrate.

6. The system of claim 5, wherein the conductive trace comprises copper.

7. The system of either of claims 5 and 6, wherein the conductive trace comprises a serpentine portion.

8. The system of any preceding claim, wherein at least one of the stretchable contacts comprises a first contact region coupled to its respective capacitive electrode.

9. The system of claim 8, wherein the coupling between the first contact region and the respective capacitive electrode comprises a bond created by melting a/the TPU substrate of the at least one of the stretchable contacts.

10. The system of either of claims 8 and 9, wherein the first contact region comprises a conductive planar surface.

11. The system of any of claims 8 to 10, wherein the first contact region is circular in shape.

12. The system of any preceding claim, wherein at least one of the stretchable contacts comprises a second contact region coupled to the PCB.

13. The system of claim 12, wherein the coupling between the second contact region and the PCB comprises a bond created by melting a/the TPU substrate of the at least one of the stretchable contacts.

14. The system of either of claim 12 or 13, wherein the second contact region comprises a conductive planar surface.

15. The system of any of claims 12 to 14, wherein the second contact region is circular in shape.

16. The system of any preceding claim, wherein at least one of the capacitive electrodes comprises first and second layers of an electrically conductive textile material separated by a layer of electrically insulating textile material.

17. The system of claim 16, wherein a cutaway is provided on the at least one of the capacitive electrodes, the cutaway comprising a region where the first layer of the electrically conductive textile material and the electrically insulating textile material have been removed, wherein the respective stretchable contact is electrically connected to the second layer of the electrically conductive textile material via the cutaway.

18. The system of any preceding claim, wherein each of the two capacitive electrodes is arranged either side of a longitudinal axis of the sheet.

19. The system of any preceding claim, wherein the system comprises four capacitive electrodes.

20. The system of claim 19, wherein the four capacitive electrodes are arranged in pairs either side of a longitudinal axis of the sheet.

21. The system of any preceding claim, wherein the PCB comprises a thermoplastic polyurethane substrate.

22. The system of any preceding claim, wherein the system further comprises an electronic signal processing system connected to each capacitive electrode via the PCB and each electrode's respective stretchable contact, wherein the electronic signal processing system is configured to process biosignals detected by the capacitive electrodes.

23. The system of any preceding claim, wherein the detected biosignals comprise ECG signals.

24. The system of any preceding claim, the system further comprising a temperature sensor supported by the sheet and in electrical contact with the PCB.

25. The system of claim 24, wherein the temperature sensor comprises an array of temperature sensors.

26. The system of any preceding claim, wherein the two capacitive electrodes are of the same size.

27. The system of any preceding claim, wherein the PCB and two stretchable contacts comprise a single substrate.

28. The system of claim 27, wherein the single substrate comprises a thermoplastic polyurethane substrate.
